(19) United States
(12) Patent Application Publication
(10) Pub. No.: US 2007/0253572 A1
(43) Pub. Date: Nov. 1, 2007

(54) SOUND REPRODUCTION DEVICE AND METHOD FOR HEARING PROTECTION IN AN AMBIENT ENVIRONMENT

(75) Inventors: Bing Li, Shenzhen (CN); Xiao-Guang Li, Shenzhen (CN); Wen-Sheng Tsai, Shenzhen (CN); Kuan-Hong Hsieh, Shenzhen (CN)

Correspondence Address: NORTH AMERICA INTELLECTUAL PROPERTY CORPORATION, P.O. BOX 506, MERRIFIELD, VA, 22116, US

(73) Assignee: HON HAI PRECISION INDUSTRY CO., LTD., Taipei Hsien (TW)

(21) Appl. No.: 11/619,619

(22) Filed: Jan. 4, 2007

(30) Foreign Application Priority Data
Apr. 19, 2006 (CN) 200610060400.7

(51) Int. Cl. A61F 11/06; H03G 3/20

(52) U.S. Cl. 381/72; 381/57

(57) ABSTRACT

The present invention relates to a method for hearing protection in an ambient environment of a sound reproduction device, includes steps of: receiving digital audio signals from an audio signal source via a connector; sampling the digital audio signals to obtain a plurality of sampled amplitude values; computing an actual audio energy of the digital audio signals within a predetermined time period setting the sampled amplitude values sampled within the predetermined time period as parameters; collecting ambient noises to compute a noise level; obtaining a reference audio energy corresponding to the noise level; comparing the actual audio energy with the reference audio energy; generating a hearing protect signal if the actual audio energy reaches the reference audio energy; and reducing a current gain value or emitting reminding information when receiving the hearing protect signal, thus, protecting users' hearing. The present invention also provides a corresponding sound reproduction device.

### BACKGROUND OF THE INVENTION

1. Field of the Invention

The present invention relates to a sound reproduction device and method for hearing protection in an ambient environment, especially to a sound reproduction device and method for evaluating noise level, and automatically adjusting a default gain value or emitting prompt information according to the noise level.

2. Description of Related Art

The continuous development of new digital technologies has made portable audio devices (such as MP3 player) become popular. When environmental noise external of the portable audio device is loud or when a favorite song is played, a user commonly increases a gain value of the portable audio device. However users all have a physiological hearing threshold, i.e., loudness discomfort level (LDL). If the user is exposed to a noise level that is larger than the user's LDL for a long time, the user's hearing may be impaired.

In order to solve the problems mentioned, there is a gain control apparatus and method available in the market. The gain control apparatus provides a noise level-gain value index. The noise level-gain value index lists a plurality of gain values corresponding to noise level ranges. The gain control apparatus collects the ambient noises, and computes a noise level of the ambient noises in a predetermined time field; obtaining a predetermined gain value corresponding to the noise level from the noise level-gain value index; adjusts a gain value to the predetermined gain value. Whereby the gain value of the gain control apparatus is changeable along with the noise level.

However, if a user is in an environment where the noise level changes frequently, the gain control apparatus will frequently change the gain value accordingly. As a result, the user will be uncomfortable. Furthermore, audio signals with different amplitudes will have different loudness at a same gain value.

Therefore, a heretofore unaddressed need exists in the industry to overcome the aforementioned deficiencies and inadequacies.

### SUMMARY OF THE INVENTION

The present invention provides a sound reproduction device and method for hearing protection while reproducing sounds in an ambient environment. The sound reproduction device and method evaluates noise level, and automatically changes a current gain value, or emitting prompt information according to the noise level.

In a first aspect of the invention, the sound reproduction device includes a connector, a DAC, a gain amplifier, and an electro-acoustic transducer. The connector is configured for attaching an audio signal source. The DAC receives digital audio signals from the audio signal source via the connector, and converts the digital audio signals to analog audio signals. The gain amplifier amplifies the analog audio signals. The electro-acoustic transducer reproduces sounds corresponding to the amplified analog audio signals. The sound reproduction device further includes a storage unit, a processing unit, a microphone, and an ADC. The microphone collects ambient noises to generate analog noise signals. The ADC converts the analog noise signals to digital noise signals. The storage unit stores a default gain value. The processing unit includes an amplitude sampling module, a gain obtaining module, an energy computing module and a noise processing module. The amplitude sampling module receives the digital audio signals from the audio signal source via the connector, and samples the digital audio signals to obtain a plurality of sampled amplitude values. The gain obtaining module obtains the default gain value from the storage unit. The energy computing module computes an actual audio energy of the digital audio signals within a predetermined time period by the default gain value and the sampled amplitude values sampled within the predetermined time period. The noise processing module computes a noise level according to the digital noise signals generated by the ADC, obtaining a reference audio energy corresponding to the noise level, comparing the actual audio energy with the reference audio energy, and generating a hearing protect signal when the actual audio energy reaches the reference audio energy.

In a second aspect of the invention, the sound reproduction device includes a connector, a DAC, and an electro-acoustic transducer. The connector is configured for attaching to an audio signal source. The DAC receives digital audio signals from the audio signal source via the connector, and converts the digital audio signals to analog audio signals. The electro-acoustic transducer reproduces sounds corresponding to the amplified analog audio signals. The sound reproduction device further includes a processing unit, a microphone, and an ADC. The microphone collects ambient noises to generate analog noise signals. The ADC converts the analog noise signals to digital noise signals. The processing unit includes an amplitude sampling module, an energy computing module and a noise processing module. The amplitude sampling module receives the digital audio signals from the audio signal source via the connector, and samples the digital audio signals to obtain a plurality of sampled amplitude values. The energy computing module computes an actual audio energy of the digital audio signals within a predetermined time period by the sampled amplitude values sampled within the predetermined time period. The noise processing module computes a noise level according to the digital noise signals generated by the ADC, obtaining a reference audio energy corresponding to the noise level, comparing the actual audio energy with the reference audio energy, and generating a hearing protect signal when the actual audio energy reaches the reference audio energy.

The hearing protection method includes the steps of: receiving digital audio signals from an audio signal source via a connector; sampling the digital audio signals to obtain a plurality of sampled amplitude values of the digital audio signals; computing an actual audio energy of the digital audio signals within a predetermined time period setting the sampled amplitude values sampled within the predetermined time period as parameters; collecting ambient noises to compute a noise level; obtaining a reference audio energy corresponding to the noise level; comparing the actual audio energy with the reference audio energy; and generating a hearing protect signal if the actual audio energy reaches the reference audio energy.

Other systems, methods, features, and advantages will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a schematic diagram of a hardware infrastructure of a sound reproduction device for hearing protection in an ambient environment in accordance with a first preferred embodiment of the present invention.

FIG. 2 is a block diagram of a hardware infrastructure of the sound reproduction device of FIG. 1 in accordance with the first preferred embodiment of the present invention;

FIG. 3 is a schematic diagram of main function modules of a processing unit of FIG. 2;

FIG. 4 is a flowchart of a preferred hearing protection method in the ambient environment by utilizing the sound reproduction device of FIG. 2;

FIG. 5 is a schematic diagram of a hardware infrastructure of a sound reproduction device for hearing protection in the ambient environment in accordance with a second preferred embodiment of the present invention;

FIG. 6 is a block diagram of a hardware infrastructure of the sound reproduction device of FIG. 5 in accordance with the second preferred embodiment of the present invention;

FIG. 7 is a schematic diagram of main function modules of a processing unit of FIG. 6; and

FIG. 8 is a block diagram of a hardware infrastructure of the sound reproduction device in accordance with a third preferred embodiment of the present invention.

### DETAILED DESCRIPTION OF THE INVENTION

In the following embodiments, for simplicity, a hearing protection function incorporated in a sound reproduction device, such as an earphone, is depicted. The sound reproduction device of the present invention allows hearing protection while reproducing sounds in an ambient environment. The following detailed description of the embodiments is made with reference to the attached drawings.

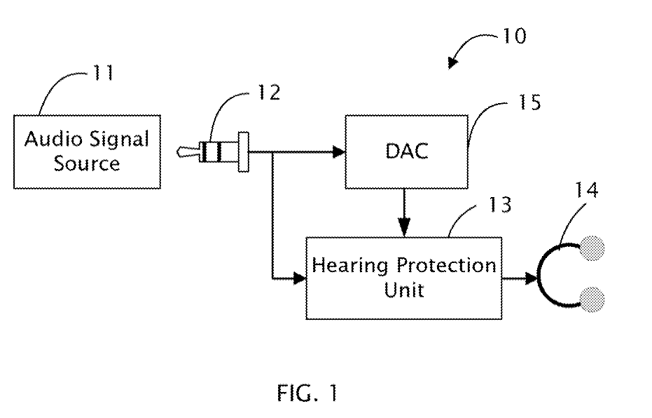

FIG. 1 is a schematic diagram of a hardware infrastructure of the sound reproduction device for hearing protection in accordance with a first preferred embodiment of the present invention. The sound reproduction device 10 includes a connector 12, a hearing protection unit 13, a digital-to-analog converter (DAC) 15, and an electro-acoustic transducer 14. The sound reproduction device 10 receives digital audio signals from an audio signal source 11 via the connector 12, and sends the digital audio signals to the hearing protection unit 13 and the DAC 15. The audio signal source 11 can be a music player, a radio player, a TV set, and so on.

The DAC 15 receives the digital audio signals from the audio signal source 11, converts the digital audio signals to analog audio signals, and sends the analog audio signals to the hearing protection unit 13.

The hearing protection unit 13 computes an audio energy of the digital audio signals within a predetermined time period, and when the audio energy reaches a predetermined value, automatically changes a default gain value to a reduced gain value.

The electro-acoustic transducer 14 receives the analog audio signals from the DAC 15, and reproduces sounds corresponding to the analog audio signals. The electro-acoustic transducer 14 may be an earphone or a speaker.

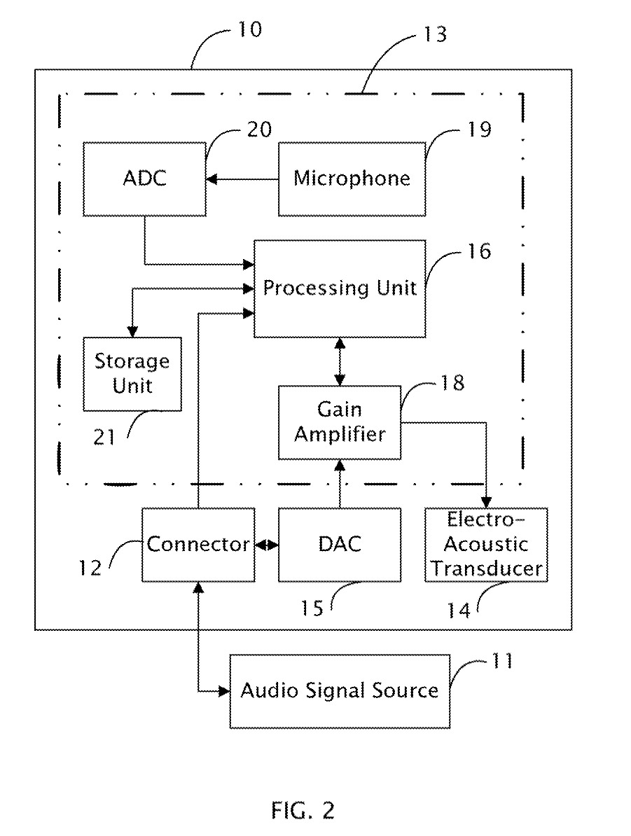

FIG. 2 is a block diagram of a hardware infrastructure of the sound reproduction device of FIG. 1 in accordance with the second preferred embodiment. The hearing protection unit 13 includes a processing unit 16, a gain amplifier 18, and a storage unit 21. The storage unit 21 stores a default gain value. The storage unit 21 may be a flash storage, a hard disk driver, and the like. The gain amplifier 18 is configured for receiving and amplifying the analog audio signals, thereby yielding amplified analog signals that is then sent to the electro-acoustic transducer 14.

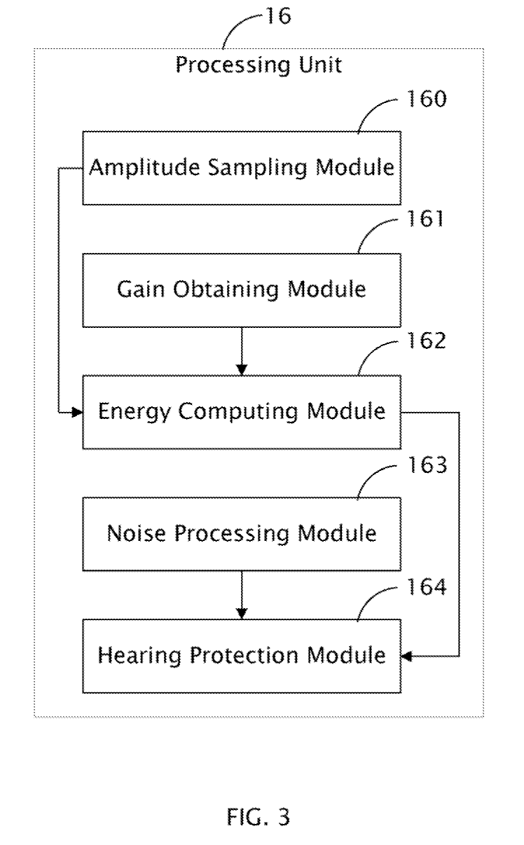

Referring to FIG. 3, the processing unit 16 includes an amplitude sampling module 160, a gain obtaining module 161, an energy computing module 162, a noise processing module 163, and a hearing protection module 164.

The amplitude sampling module 160 receives the digital audio signals from the audio signal source 11 via the connector 12, samples the digital audio signals at a predetermined frequency to obtain a plurality of sampled amplitude values of the digital audio signals. The gain obtaining module 161 obtains the default gain value from the storage unit 21.

The energy computing module 162 computes an actual audio energy of the digital audio signals within the predetermined time period by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

The hearing protection unit 13 further includes a microphone 19 and an analog-to-digital converter (ADC) 20. The microphone 19 is configured for collecting ambient noises, and for generating analog noise signals according to the ambient noises. The ADC 20 is configured for converting the analog noise signals to digital noise signals.

The noise processing module 163 computes a noise level according to the digital noise signals, and computes a reference audio energy corresponding to the noise level. In a first preferred method, the noise processing module 163 subtracts a predetermined noise level from the noise level to obtain a margin, divides the margin by the predetermined noise level to obtain the changed ratio, i.e., changed ratio=(noise level−predetermined noise level)/predetermined noise level, and multiplies the changed ratio with a predetermined audio energy to obtain the reference audio energy.

In an alternative preferred method, the storage unit 21 further stores a noise level-audio energy index. The noise level-audio energy index is a table that lists the reference audio energy corresponding to noise level ranges. The noise processing module 163 searches the noise level-audio energy index with the noise level to obtain the reference audio energy correspondingly.

The noise processing module 163 compares the actual audio energy with the reference audio energy, and generates a hearing protect signal if the actual audio energy reaches the reference audio energy. The reference audio energy is an upper threshold value (loudest sound intensity) appropriate for a listener.

When the hearing protection module 164 receives the hearing protect signal, the hearing protection module 164 automatically changes the default gain value to the reduced gain value, signals the gain amplifier 18 to amplify the analog audio signals received from the DAC 15 with the reduced gain value, and updates the default gain value in the storage unit 21 with the reduced gain value.

In an alternative preferred embodiment, when the hearing protection module 164 receives the hearing protect signal, the hearing protection module 164 sends prompt signals to the gain amplifier 18. The gain amplifier 18 is configured for receiving and amplifying the prompt signals, thereby yielding amplified prompt signals that is then sent to the electro-acoustic transducer 14. The electro-acoustic transducer 14 reproduces prompt sounds according to the amplified prompt signals. The prompt sounds are used for alerting the listener to reduce the current gain value of the audio signal source 11.

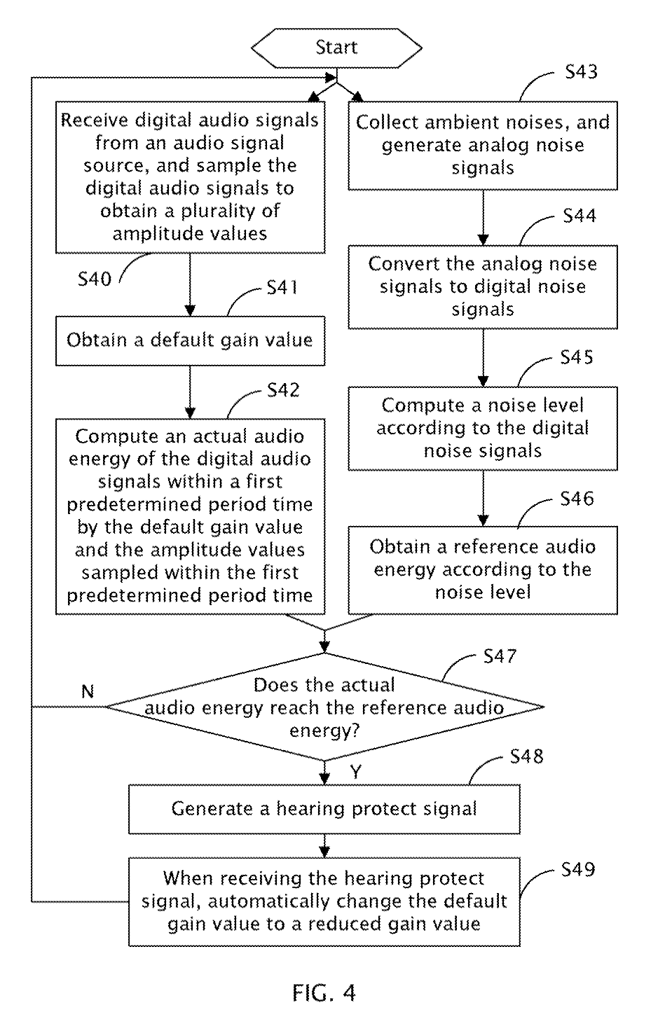

FIG. 4 is a flowchart of a first preferred method for hearing protection in the ambient environment by utilizing the sound reproduction device of FIG. 2. In step S40, the amplitude sampling module 160 receives the digital audio signals from the audio signal source 11 via the connector 12, and samples the digital audio signals at the predetermined frequency to obtain the plurality of sampled amplitude values of the digital audio signals.

In step S41, the gain obtaining module 161 obtains the default gain value from the storage unit 21.

In step S42, the energy computing module 162 computes the actual audio energy of the digital audio signals within the predetermined time period by the default gain value and the sampled amplitude values within the predetermined time period.

Simultaneously with step S42, in step S43, the microphone 19 collects the ambient noises from the ambient environment, and generates the analog noise signals.

In step S44, the ADC 20 converts the analog noise signals to the digital ambient noises.

In step S45, the noise processing module 163 computes the noise level according to the digital noise signals converted by the ADC 20.

In step S46, the noise processing module 163 reads the reference audio energy corresponding to the noise level from the noise level-audio energy index, or alternatively, computes the reference audio energy.

In step S47, the noise processing module 163 compares the actual audio energy with the reference audio energy, detects whether the actual audio energy reaches the reference audio energy. If the actual audio energy does not reach the reference audio energy, the procedure returns to start.

When the actual audio energy reaches the reference audio energy, in step S48, the noise processing module 163 generates the hearing protect signal.

In step S49, when receiving the hearing protect signal, the hearing protection module 164 automatically changes the default gain value to the reduced gain value, signals the gain amplifier 18 to amplify the analog audio signals generated by the reduced gain value, and updates the default gain value stored in the storage unit 21 with the reduced gain value, then the procedure returns to start.

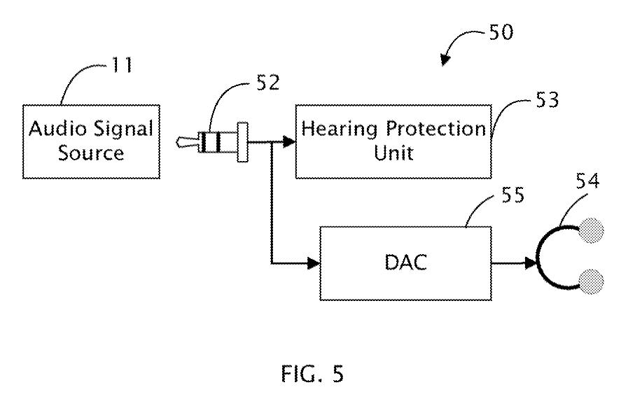

FIG. 5 is a schematic diagram of a hardware infrastructure of the sound reproduction device for hearing protection in accordance with a second preferred embodiment of the present invention. The sound reproduction device 50 includes a connector 52, a hearing protection unit 53, a DAC 55, and an electro-acoustic transducer 54. The sound reproduction device 50 receives the digital audio signals from the audio signal source 11 via the connector 52, and sends the digital audio signals to the hearing protection unit 53 and the DAC 55.

The hearing protection unit 53 computes an audio energy of the digital audio signals within the predetermined time period, and when the audio energy reaches the predetermined value, outputs a prompt indicator to alert the listener to manually reduce the current gain value of the audio signal source, thus, preventing hearing impairment of the user. The prompt indicator is selected from the group consisting of visual indicator and acoustical indicator.

The DAC 55 converts the digital audio signals received from the audio signal source 11 to analog audio signals, and sends the analog audio signals to the hearing protection unit 53.

The electro-acoustic transducer 54 reproduces sounds corresponding to the analog audio signals generated by the DAC 55. The electro-acoustic transducer 54 may be an earphone or a speaker.

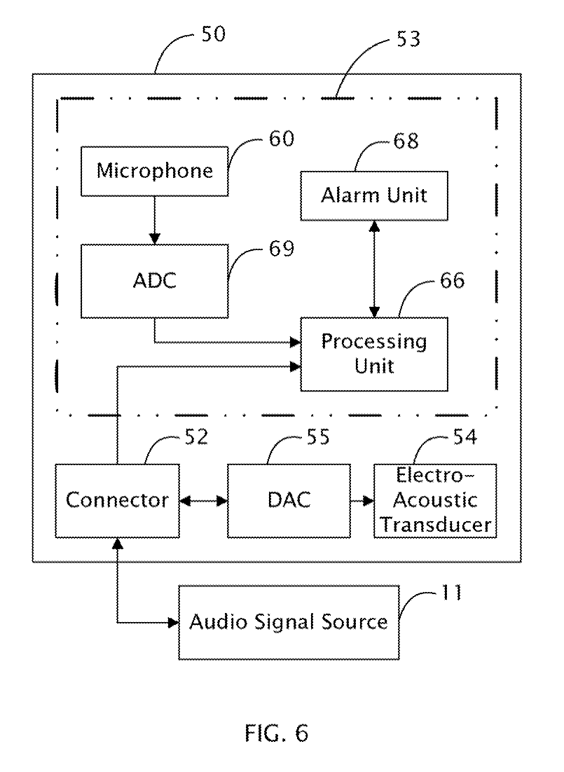

FIG. 6 is a block diagram of a hardware infrastructure of the sound reproduction device 50 of FIG. 5 in accordance with the second preferred embodiment. The hearing protection unit 53 includes a processing unit 66, an alarm unit 68, a storage unit 61, a microphone 60, and an ADC 69. The alarm unit 68 may be an acoustical indicating device such as a buzzer, or a visual indicating device such as an LED (light-emitting diode).

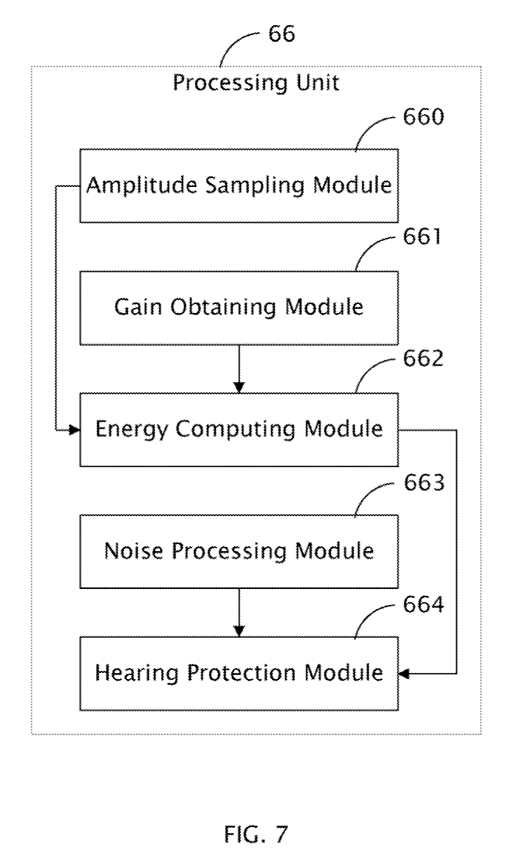

Referring to FIG. 7, the processing unit 66 includes an amplitude sampling module 660, a gain obtaining module 661, an energy computing module 662, a noise processing module 663, and a hearing protection module 664.

The amplitude sampling module 660 receives the digital audio signals from the audio signal source 11 via the connector 52, samples the digital audio signals at the predetermined frequency, and obtains the plurality of sampled amplitude values of the digital audio signals.

The energy computing module 661 periodically computes an audio energy within the predetermined time period by: Q=[Σ(mi)²/N]^{1/2}*T, wherein Q represents the actual audio energy, mi presents the sampled amplitude values sampled within the predetermined time period, N represents the count of the sampled amplitude values sampled within the predetermined time period, and i denotes an identifier of the sampled amplitude value.

The hearing protection unit 63 further includes a microphone 60 and an ADC 69. The microphone 60 is configured for collecting ambient noises, and generating analog noise signals. The ADC 60 is configured for converting the analog noise signals to the digital noise signals.

The noise processing module 663 computes the noise level according to the digital noise signals, and obtains the reference audio energy corresponding to the noise level. In a preferred method, the noise processing module 663 subtracts the predetermined noise level from the noise level to obtain the margin, divides the margin by the predetermined noise level to obtain the changed ratio, i.e., changed ratio=(noise level−predetermined noise level)/predetermined noise level, and multiplies the changed ratio with the predetermined audio energy to obtain the reference audio energy.

In an alternative preferred method, the hearing protection unit 63 further stores a storage unit 61 for storing the noise level-audio energy index. The noise level-audio energy index is the table that lists the reference audio energy corresponding to noise level ranges. The noise processing module 663 reads the noise level-audio energy index to obtain the reference audio energy corresponding to the noise level.

The noise processing module 663 compares the actual audio energy with the reference audio energy, and generates a hearing protect signal when the actual audio energy reaches the reference audio energy.

The hearing protection module 664, when receiving the hearing protect signal, signals the alarm unit 78 to output prompt information. The prompt information is used for alerting the user to manually reduce the current gain value of the audio signal source 11, thus, preventing hearing impairment of the user.

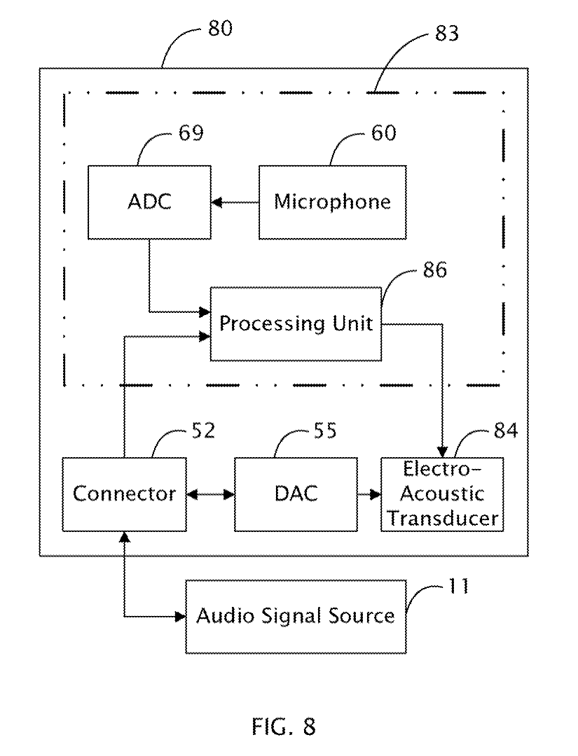

In a third preferred embodiment, referring to FIG. 8, the difference between the third embodiment and the second embodiment is that the hearing protection unit 83 of the third embodiment does not include the alarm unit 68, and a processing unit 86 and an electro-acoustic transducer 84 is adopted to perform the same functions of the alarm unit 68.

The processing unit 86 includes an amplitude sampling module 860, a gain obtaining module 861, an energy computing module 862, and a noise processing module 863, which respectively performs the same functions as the amplitude sampling module 160, the gain obtaining module 161, the energy computing module 162, and the noise processing module 163 of the processing unit 16 in the first and second embodiment.

The processing unit 86 further includes a hearing protection module 864. The hearing protection module 864, when receiving the hearing protect signal, sends the prompt signals to the electro-acoustic transducer 84. The electro-acoustic transducer 84 outputs prompt sounds corresponding to the prompt signals.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. A sound reproduction device for hearing protection in an ambient environment, the sound reproduction device comprising:
a connector configured for attaching to an audio signal source;
a DAC configured for receiving digital audio signals from the audio signal source via the connector, and converting the digital audio signals to analog audio signals;
a gain amplifier configured for receiving and amplifying the analog audio signals, thereby yielding amplified analog audio signals;
an electro-acoustic transducer configured for reproducing sounds corresponding to the analog audio signals;
a microphone configured for collecting ambient noises, and generating analog noise signals;
an ADC configured for converting the analog noise signals to digital noise signals;
a storage unit configured for storing a default gain value; and
a processing unit comprising:
an amplitude sampling module for receiving the digital audio signals from the audio signal source via the connector, and sampling the digital audio signals to obtain a plurality of sampled amplitude values;
a gain obtaining module for obtaining the default gain value from the storage unit;
an energy computing module for computing an actual audio energy of the digital audio signals within a predetermined time period by the default gain value and the sampled amplitude values sampled within the predetermined time period; and
a noise processing module for computing a noise level according to the digital noise signals generated by the ADC, obtaining a reference audio energy corresponding to the noise level, comparing the actual audio energy with the reference audio energy, and generating a hearing protect signal if the actual audio energy reaches the reference audio energy.

2. The sound reproduction device according to claim 1, wherein the processing unit further comprises a hearing protection module, when receiving the hearing protect signal, the hearing protection module automatically changes the default gain value to a reduced gain value, signals the gain amplifier to amplify the analog audio signals received from the DAC by the reduced gain value, and updates the default gain value stored in the storage unit with the reduced gain value.

3. The sound reproduction device according to claim 1, wherein the processing unit further comprises a hearing protection module, when receiving the hearing protect signal, the hearing protection module sends prompt signals to the gain amplifier, the gain amplifier amplifies the prompt signals, and the electro-acoustic transducer reproduces prompt sounds corresponding to the amplified prompt signals.

4. The sound reproduction device according to claim 1, wherein:
the storage unit further stores a noise level-audio energy index for listing reference audio energy corresponding to noise level ranges; and
the noise processing module reads the noise level-audio energy index to obtain the reference audio energy corresponding to the noise level.

5. The sound reproduction device according to claim 1, wherein the noise processing module subtracts a predetermined noise level from the noise level to obtain a margin, divides the margin by the predetermined noise level to get a changed ratio, and multiplies a predetermined audio energy by the changed ratio to obtain the reference audio energy.

6. The sound reproduction device according to claim 1, wherein the actual audio energy is computed by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

7. A sound reproduction device for hearing protection in an ambient environment, the sound reproduction device comprising:
a connector configured for attaching to an audio signal source;
a DAC configured for receiving digital audio signals from the audio signal source via the connector, and converting the digital audio signals to analog audio signals;
an electro-acoustic transducer configured for reproducing sounds corresponding to the analog audio signals;
a microphone configured for collecting ambient noises, and generating analog noise signals;
an ADC configured for converting the analog noise signals to digital noise signals; and
a processing unit comprising:
an amplitude sampling module for receiving the digital audio signals from the audio signal source via the connector, and sampling the digital audio signals to obtain a plurality of sampled amplitude values;
an energy computing module for computing an actual audio energy of the digital audio signals within a predetermined time period by the sampled amplitude values sampled within the predetermined time period; and
a noise processing module for computing a noise level according to the digital noise signals generated by the ADC, obtaining a reference audio energy corresponding to the noise level, comparing the actual audio energy with the reference audio energy, generating a hearing protect signal if the actual audio energy reaches the reference audio energy.

8. The sound reproduction device according to claim 7, wherein the processing unit further comprises a hearing protection module, when receiving the hearing protect signal, the hearing protection module sends prompt signals to the electro-acoustic transducer, and the electro-acoustic transducer outputs prompt sounds corresponding to the prompt signals.

9. The sound reproduction device according to claim 7, further comprising an alarm unit, wherein the processing unit further comprises a hearing protection module, when receiving the hearing protect signal, the hearing protection module signals the alarm unit to output prompt information.

10. The sound reproduction device according to claim 9, wherein the prompt information is selected from the group consisting of visual reminding information and acoustical reminding information.

11. The sound reproduction device according to claim 7, further comprising a storage unit, wherein the storage unit stores a noise level-audio energy index for listing reference audio energy corresponding to noise level ranges; and the noise processing module reads the noise level-audio energy index to obtain the reference audio energy corresponding to the noise level.

12. The sound reproduction device according to claim 7, wherein the noise processing module subtracts a predetermined noise level from the noise level to obtain a margin, divides the margin by the predetermined noise level to get a changed ratio, and multiplies a predetermined audio energy by the changed ratio to obtain the reference audio energy.

13. The sound reproduction device according to claim 7, wherein the actual audio energy is computed by: Q=[Σ(mi)²/N]^{1/2}, wherein Q represents the actual audio energy, mi represents the sampled amplitude values sampled within the predetermined time period, N represents a count of the sampled amplitude values sampled within the predetermined time period, and i denotes an identifier of the sampled amplitude value.

14. A hearing protection method in an ambient environment of a sound reproduction device, the method comprising the steps of:
receiving digital audio signals from an audio signal source via a connector;
sampling the digital audio signals to obtain a plurality of sampled amplitude values of the digital audio signals;
computing an actual audio energy of the digital audio signals within a predetermined time period setting the sampled amplitude values sampled within the predetermined time period as parameters;
collecting ambient noises to compute a noise level;
obtaining a reference audio energy corresponding to the noise level;
comparing the actual audio energy with the reference audio energy; and
generating a hearing protect signal if the actual audio energy reaches the reference audio energy.

15. The hearing protection method according to claim 14, wherein the actual audio energy is computed by: Q=[Σ(mi)²/N]^{1/2}, wherein Q represents the actual audio energy, mi represents the sampled amplitude values sampled within the predetermined time period, N represents a count of the sampled amplitude values sampled within the predetermined time period, and i denotes an identifier of the sampled amplitude value.

16. The hearing protection method according to claim 14, further comprising the step of:
obtaining a default gain value acting as a parameter for computing the actual audio energy by: Q=[Σ(mi*V)²/N]^{1/2}, wherein Q represents the actual audio energy, V presents the default gain value, mi presents the sampled amplitude values sampled within the predetermined time period, N represents a count of the sampled amplitude values sampled within the predetermined time period, and i denotes an identifier of the sampled amplitude value.

17. The hearing protection method according to claim 16, further comprising the steps of:
when receiving the hearing protect signal, automatically reducing the default gain value to a reduced gain value, converting the digital audio signals to analog audio signals, amplifying the analog audio signals by the reduced gain value, and updating the default gain value with the reduced gain value.

18. The hearing protection method according to claim 14, wherein the step of obtaining the reference audio energy comprises the steps of:
providing a storage unit for storing a noise level-audio energy index, the noise level-audio energy index listing reference audio energy corresponding to noise level ranges; and
reading the noise level-audio energy index to obtain the reference audio energy corresponding to the noise level.

19. The hearing protection method according to claim 14, wherein the step of obtaining the reference audio energy comprises the steps of:
subtracting a predetermined noise level from the noise level to obtain a margin;
dividing the margin by the predetermined noise level to get a changed ratio; and
multiplying a predetermined audio energy by the changed ratio to obtain the reference audio energy.

20. The hearing protection method according to claim 14, further comprising the steps of:
when receiving the hearing protect signal, emitting prompt information.

21. The hearing protection method according to claim 20, wherein the prompt information is selected from the group consisting of visual reminding information and acoustical reminding information.
